(19) United States
(12) Patent Application Publication
(10) Pub. No.: US 2025/0345172 A1
(43) Pub. Date: Nov. 13, 2025

(54) Polymeric Fetal Heart Valve Devices and Methods of Making Same

(71) Applicants: Georgia Tech Research Corporation, Atlanta, GA (US); Nationwide Children's Hospital, Columbus, OH (US)

(72) Inventors: Hieu Bui, Atlanta, GA (US); Sanchita Satish Bhat, Atlanta, GA (US); Lakshmi Prasad Dasi, Atlanta, GA (US); Aimee K. Armstrong, Columbus, OH (US); Christopher K. Breuer, Columbus, OH (US)

(21) Appl. No.: 18/862,606

(22) PCT Filed: May 4, 2023
(86) PCT No.: PCT/US2023/066594

§ 371 Date: Nov. 4, 2024

Related U.S. Application Data
(60) Provisional application No. 63/364,129, filed on May 4, 2022.

(51) Int. Cl. A61F 2/24; B29C 39/10; B29C 39/26; B29C 39/36; B29L 31/00

(52) U.S. Cl. CPC A61F 2/2415; B29C 39/10; B29C 39/26; B29C 39/36; A61F 2210/0004; B29L 2031/7534

(57) ABSTRACT

An exemplary embodiment of the present disclosure provides a method for producing a resorbable heart valve, the method comprising: providing a first solution comprising a polymeric material; coating at least a portion of a mold with the first solution for form a coating; forming a plurality of valve leaflets on a first end of the coating; placing a frame around at least a portion of the coating; and attaching the frame to the coating.

### CROSS-REFERENCE TO RELATED APPLICATIONS

This application claims the benefit of U.S. Provisional Application Ser. No. 63/364,129, filed on 4 May 2022, which is incorporated herein by reference in its entirety as if fully set forth below.

### FIELD OF THE DISCLOSURE

The various embodiments of the present disclosure relate generally to heart valves and methods of their manufacture.

### BACKGROUND

Single ventricle heart disease is a rare type of congenital heart defect affecting about five out of 10,000 newborns. Traditionally, this is surgically palliated in three stages. Despite these procedures, the morbidity and mortality are high, and most will require heart transplantation. Some fetuses with developing single ventricle heart disease are candidates for percutaneous transcatheter balloon valvuloplasty of the pulmonary valve or aortic valve in an attempt to prevent the development of hypoplastic right or left heart syndrome, respectively. If the fetal intervention prevents single ventricle disease, the prognosis is thought to be improved, but the pulmonary or aortic valve ultimately needs to be replaced as restenosis or regurgitation develops. If this is done in childhood, the prosthetic valve often requires replacement due to somatic growth of the child, as well as degeneration of the valve, which can be hastened in a growing child. For instance, the average time to a second right ventricular to pulmonary artery valved conduit replacement is 7.5 years. A bioresorbable tissue-engineered valve that could be replaced by the patient's own tissue could allow the valve to grow with the patient and could preclude the need for multiple valve replacements during a patient's lifetime. A goal of a resorbable valve is for the tissue-engineered scaffold to serve as a template to direct tissue formation. As the scaffold degrades, the neotissue can form, ultimately creating a living autologous valve. Fortunately, recent advancements have demonstrated the efficacy of a fully resorbable tissue-engineered fetal valve design with the help of a fetal ovine mode. Due to the transcatheter nature of this valve deployment, the procedure may not be riskier than a traditional fetal valvuloplasty. However, restoration of normal valve function in utero holds the potential to restore biventricular anatomy and function.

### BRIEF SUMMARY

An exemplary embodiment of the present disclosure provides a method for producing a resorbable heart valve, the method comprising: providing a first solution comprising a polymeric material; coating at least a portion of a mold with the first solution to form a coating; forming a plurality of valve leaflets on a first end of the coating; placing a frame around at least a portion of the coating; and attaching the frame to the coating.

In any of the embodiments disclosed herein, coating the at least a portion of the mold with the first solution can comprise spray coating the at least a portion of the mold with the first solution.

In any of the embodiments disclosed herein, coating the at least a portion of the mold with the first solution can comprise airbrushing the at least a portion of the mold with the first solution.

In any of the embodiments disclosed herein, coating the at least a portion of the mold with the first solution can comprise dip coating the at least a portion of the mold with the first solution.

In any of the embodiments disclosed herein, the polymeric material can comprise a synthetic polymer.

In any of the embodiments disclosed herein, the synthetic polymer can be selected from the group consisting of polyethers, polyamides, polyurethanes, and polyesters.

In any of the embodiments disclosed herein, the polymeric material further comprises a naturally derived polymer.

In any of the embodiments disclosed herein, the naturally derived polymer can be selected from the group consisting of polypeptides, proteins, polysaccharides, glycoproteins, and glycosaminoglycan.

In any of the embodiments disclosed herein, the polymeric material can comprise a naturally derived polymer.

In any of the embodiments disclosed herein, the naturally derived polymer can be selected from the group consisting of polypeptides, proteins, polysaccharides, glycoproteins, and glycosaminoglycan.

In any of the embodiments disclosed herein, the polymeric material can comprise at least one biodegradable polymeric material.

In any of the embodiments disclosed herein, the coating can be a multi-layered coating, and coating the at least a portion of the mold with the first solution can form a first layer in the multi-layered coating.

In any of the embodiments disclosed herein, the method can further comprise coating the at least a portion of the first layer with a second solution to form a second layer of the multi-layer coating.

In any of the embodiments disclosed herein, the plurality of leaflets can comprise one or more commissures, and the commissures can have a thickness greater than an average thickness of the plurality of leaflets.

In any of the embodiments disclosed herein, attaching the frame to the coating can comprise suturing the frame to the coating.

In any of the embodiments disclosed herein, attaching the frame to the coating can comprise coating at least a portion of the frame with a third solution.

In any of the embodiments disclosed herein, the third solution can be different than the first solution.

In any of the embodiments disclosed herein, the frame can be configured as an expandable and contractable stent.

In any of the embodiments disclosed herein, the expandable and contractable stent can comprise a plurality of struts.

In any of the embodiments disclosed herein, the frame can comprise a material selected from the group consisting of plastics, metals, and carbon.

In any of the embodiments disclosed herein, the frame can comprise a metal selected from the group consisting of zinc, iron, aluminum, magnesium, nickel, silver, titanium, and alloys thereof.

In any of the embodiments disclosed herein, the frame can comprise a bioresorbable material.

Another embodiment of the present disclosure provides a resorbable heart valve. The heart valve can comprise a frame and a plurality of leaflets coupled to the frame. The plurality of leaflets can comprise a first polymeric material.

In any of the embodiments disclosed herein, the plurality of leaflets can be multi-layered.

In any of the embodiments disclosed herein, a first layer in the multi-layered leaflets can comprise a synthetic polymer.

In any of the embodiments disclosed herein, a second layer in the multi-layered leaflets can comprise a naturally derived polymer.

In any of the embodiments disclosed herein, the valve can comprise one or more sutures coupling the frame to the plurality of leaflets.

In any of the embodiments disclosed herein, the frame can be attached to the plurality of leaflets with a second polymeric material.

In any of the embodiments disclosed herein, the second polymeric material can be different than the first polymeric material.

Another embodiment of the present disclosure provides a device for use in implanting a valve in a patient. The device can comprise a cannula, a trocar, and a catheter. The cannula can have a first end, a second end, and an outer wall defining an internal cavity. The trocar can be positioned within the internal cavity of the cannula. The trocar can have an outer wall defining an internal cavity. The catheter can be positioned within the internal cavity of the trocar.

In any of the embodiments disclosed herein, the trocar can comprise a sharpened tip comprising an opening into the internal cavity of the trocar.

In any of the embodiments disclosed herein, the opening in the sharpened tip of the trocar can be asymmetrical.

In any of the embodiments disclosed herein, the device can further comprise a pressure transducer configured to monitor a pressure within a portion of the internal cavity of the trocar.

In any of the embodiments disclosed herein, the pressure transducer can be disposed within the internal cavity of the trocar.

In any of the embodiments disclosed herein, the device can further comprise one or more circumferential perforations extending through the outer wall of the trocar and into the catheter.

In any of the embodiments disclosed herein, the outer wall of the cannula can comprise a polymeric material.

In any of the embodiments disclosed herein, the outer wall of the trocar can comprise a metal.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-D illustrate simulation results from an exemplary fetal heart valve showing (FIG. 1A) initial time point, (FIG. 1B) final time point, (FIG. 1C) en face view of final time point, and (FIG. 1D) area of maximum stress with magnitude.

FIGS. 2A-C illustrate simulation results from an exemplary fetal heart valve with 3D leaflets showing (FIG. 1A) final time point, (FIG. 1B) en face view of final time point, and (FIG. 1C) area of maximum stress with magnitude.

FIG. 3 provides a flow chart for a method of making a resorbable heart valve, in accordance with an exemplary embodiment of the present disclosure.

FIG. 4 illustrates a method of manufacturing a resorbable heart valve, in accordance with an exemplary embodiment of the present disclosure.

FIG. 5A illustrates an example of a dip coated, in accordance with an exemplary embodiment of the present disclosure. Uniaxial monotonic tensile test was performed on the dip coated leaflet material at different density, and the tensile strength and elastic modulus were calculated from regression curves as shown in FIG. 5B and FIG. 5C, respectively.

FIGS. 6A-B illustrates the en face (FIG. 6A) and isometric (FIG. 6B) views of a polymeric fetal heart valve, in accordance with an exemplary embodiment of the present disclosure.

FIG. 7 illustrates a pulsatile flow setup that was used to assess the hemodynamics of fetal valves shown in FIGS. 6A-B.

FIGS. 8A-B illustrates data obtained from two different versions of the polymeric fetal heart valve in FIG. 6. In FIG. 8A, the valve was spray-coated with 1.5 ml of polymeric solution, while, in FIG. 8B, the valve was spray-coated with 1.25 ml of the solution, in which the difference in valve performance is depicted in the bottom images, which show the valve opening at different phases throughout the cardiac cycle.

FIG. 9 illustrates a device that can be used to implant a heart valve, in accordance with some embodiments of the present disclosure.

### DETAILED DESCRIPTION

To facilitate an understanding of the principles and features of the present disclosure, various illustrative embodiments are explained below. Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

Synthetic polymers are known for their tunability and low cost. Studies have shown that synthetic polymers can be mass-produced for biomedical usages. Indeed, polymeric materials have been long used for cardiovascular applications such as heart valve repairs and replacement. Combined with the latest in utero procedures, cutting-edge polymeric valvular design may help restore healthy hemodynamics and mitigate many of the problems arising from congenital heart defects. As described below, certain embodiments of the present disclosure can make use of such synthetic polymers.

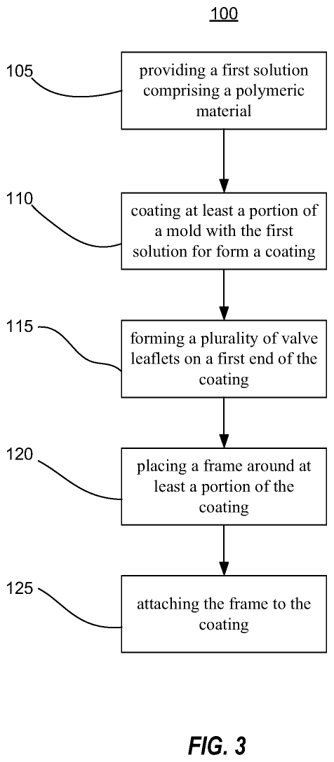

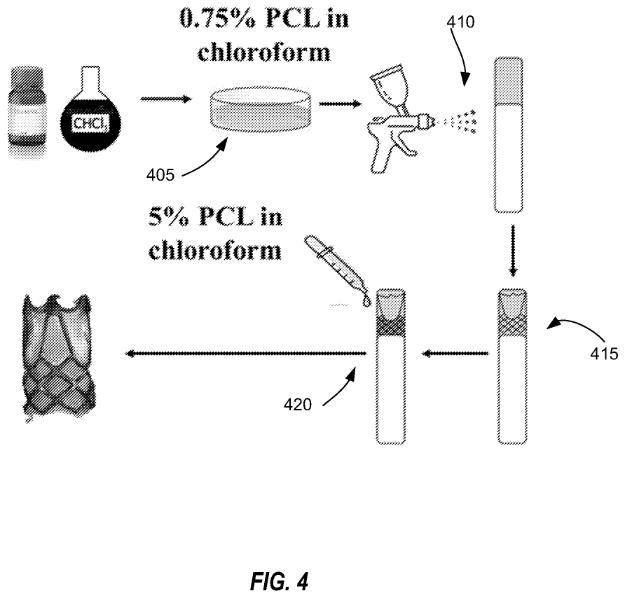

Certain embodiments of the present disclosure provide methods of fabricating replacement heart valves for use in the human fetus. As shown in FIG. 3, an exemplary embodiment of the present disclosure provides a method for producing a resorbable heart valve 100. The method can comprise: providing a first solution comprising a polymeric material 105 (illustrated at 405 in FIG. 4); coating at least a portion of a mold with the first solution for form a coating 110 (illustrated at 410 in FIG. 4); forming a plurality of valve leaflets on a first end of the coating 115; placing a frame around at least a portion of the coating 120 (illustrated at 415 in FIG. 4); and attaching the frame to the coating 125 (illustrated at 420 in FIG. 4). Once the frame has been attached to the coating, the valve (combination frame and coating) can be removed from the mold for insertion into a user (exemplary transcatheter method discussed below).

The first solution can be coated onto a portion of the mold many different ways. In some embodiments, coating the at least a portion of the mold with the first solution can comprise spray coating (or air brushing) the at least a portion of the mold with the first solution (top option for 410 in FIG. 4). In some embodiments, coating the at least a portion of the mold with the first solution can comprise dip coating the at least a portion of the mold with the first solution (bottom option for 410 in FIG. 4).

The valve can comprise multiple parts, including leaflet, stent, sutures, and skirts, each of which can comprise many different materials. The polymeric material used in the first solution deposited on the mold, which forms the valve leaflets, can be many different polymers. In some embodiments, the polymer material can be biodegradable. In some embodiments, the polymeric material can comprise one or more synthetic polymers, including, but not limited to, polyethers, polyamides, polyurethanes, and polyesters. Typical examples are polylactide, polyglycolide, polycaprolactone, polyketones, and polyethylene glycol. The leaflets can also be enhanced with naturally derived polymers, including, but not limited to, polypeptides, proteins, polysaccharides, glycoproteins, and glycosaminoglycan. Examples include arginylglycylaspartic acid, elastin, collagen, dextran, heparin, and hyaluronic acid.

The valve frames can be made from many different materials, including, but not limited to metals (including alloys thereof), polymers/plastics, and carbon. For example, some frames can comprise metal alloys that may contain elements such as zinc, iron, aluminum, magnesium, nickel, silver, titanium, and carbon. These metals can be selected due to their low toxicity and bioabsorbable nature. Examples of alloys are zinc-magnesium and zinc-aluminum. The valve frame can also comprise polymers similar to the leaflets as mentioned above, including polyethylene, polypropylene, and polytetrafluoroethylene.

For transcatheter heart valve applications, the frame can be configured as an expandable and contractable stent comprising a plurality of struts, as shown in FIG. 4. This can allow the frame to be capable of expanding and contracting to fit the size of the fetal blood vessels and the catheter deployment system (discussed below). The expanded valve can be smaller than 10 mm (outer diameter) and bigger than 4 mm (outer diameter), though, as those skilled in the art would appreciate, the disclosure is not limited to valves of this size, but rather includes many different valve sizes.

The valve and the leaflets may compose of multiple materials as described earlier (synthetic & naturally derived polymers). The leaflets can have a unique architecture (topography, hierarchy, orientation, etc.) and physical properties (porosity, density, mechanical properties, etc.) to promote hemocompatibility, durability, mechanical support, and diffusion. For example, the leaflet can have layers of distinct materials with distinct physical properties, each with different purposes. Thus, the leaflets can be formed from multi-layerd coatings. One of the layers can be a synthetic polymer for structural integrity, while another can be a naturally derived material to promote tissue ingrowth; these layers can be created following techniques described in FIGS. 3-4. For example, coating the at least a portion of the mold with the first solution 110 can form a first layer in the multi-layered coating (e.g., a synthetic polymer). The method 100 can further comprise coating the at least a portion of the first layer with a second solution to form a second layer of the multi-layer coating (e.g., a naturally derived polymer). The valve and its leaflets can also support native cells and extracellular matrix to allow tissue ingrowth.

As discussed above, the polymeric material used to create the leaflets can comprise biodegradable polymers. The leaflet material can have the capacity to degrade over time, owing to increasing shear from the incoming improved blood flow. Ideal scaffolds can be selected to maintain their properties in the time required for their eventual function.

To improve the structural stability and decrease the degradation rate in certain regions, the leaflets can be made thicker at the commissures, enhancing the leaflet attachment to the stent and overall stability during high flow. For example, in some embodiments, the commissures of the valve can have a thickness greater than an average thickness of the plurality of leaflets. The stent struts can also be designed in such a way that the commissures and the annular attachments are thicker, taking longer to degrade than the other regions. Biodegradable polymers can be highly responsive to hydrolytic degradation. Spatial degradation of the leaflet material can also be controlled this way; the heterogeneity in thickness and/or density can ensure that the free edge of the leaflet degrades quicker, while the thicker belly and commissural region take longer.

Attaching the frame to the coating 125 can be performed many different ways. In some embodiments, the frame can be sutured to the coating (leaflets). In some embodiments, a coating can be applied to the frame after the frame is positioned over the coated mold. The coating can be made of many different materials. In some embodiments, the coating can comprise the same (or one of the same) polymeric material solution(s) coated onto the mold to form the leaflets. In some embodiments, a different coating material can be used to attach the frame to the coated mold (forming the leaflets).

Described herein is also a transcatheter, percutaneous method to implant the tissue-engineered valve/stent complex into the fetal pulmonary annulus, which can use ultrasound guidance. The valve/stent complex can be manually crimped onto a balloon on the end of a catheter. A coronary wire can be placed through the balloon catheter. A 15-19-gauge cannula with trocar can be guided through the maternal abdominal wall, uterine wall, fetal chest wall, and into the right ventricle. The trocar can be removed and replaced with the wire/balloon/valve/stent complex, which can be advanced across the pulmonary annulus by ultrasound guidance. Once the stent is seen across the pulmonary annulus, it can be implanted by inflating the balloon with an inflation device. The balloon can then be deflated. The wire, catheter, balloon, and cannula can be removed, leaving the valve/stent complex implanted in the pulmonary annulus.

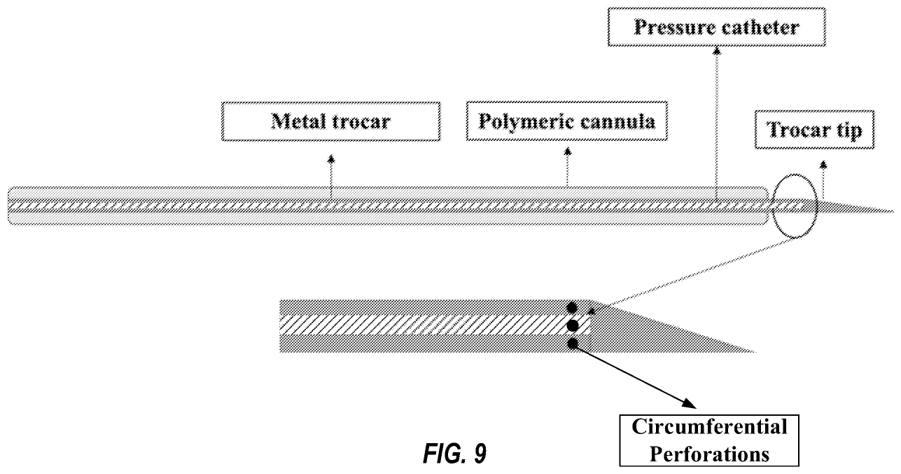

This method can be successful under the guidance of ultrasound, however, it also presents challenges. There is often blood loss from the movement of the catheter and trocar into and out of the fetal chest wall, as well as the uterine wall. There is also some uncertainty associated with the deployment, as ultrasound guided access through this complex geometry can require high level expertise. If the fetus changes position, the system may need to be recalibrated and reassessed. Therefore, some embodiments of the present disclosure provide an alternative deployment procedure to deliver the valve safely and accurately. Additionally, to supplement the currently ultrasound guided procedure, devices used for implantation can include a region in the trocar that is coupled with a pressure transducer. The system can also be MRI compatible, to allow for superior imaging to guide the deployment procedure. This can benefit across all populations receiving interventional therapies. This can guide the cardiologist on the location of the trocar, as corresponding to the pressures sensed by the proximal end, as illustrated in FIG. 9.

As shown in FIG. 9, the trocar can have circumferential perforations where small amounts of blood can be sensed with a highly sensitive pressure catheter. The polymeric cannula, in which the trocar is cased, can be constructed from a material that is compliant (to allow for movement of the trocar and then the valve) but can also have an asymmetrical opening toward the trocar. This will ensure its immediate collapse after the valve is deployed, limiting the amount of retrograde blood loss. This technique can be applied to control blood loss for other ultrasound guided techniques, in both pediatric and adult patients

### EXAMPLES

The following examples are provided by way of illustration but not by way of limitation.

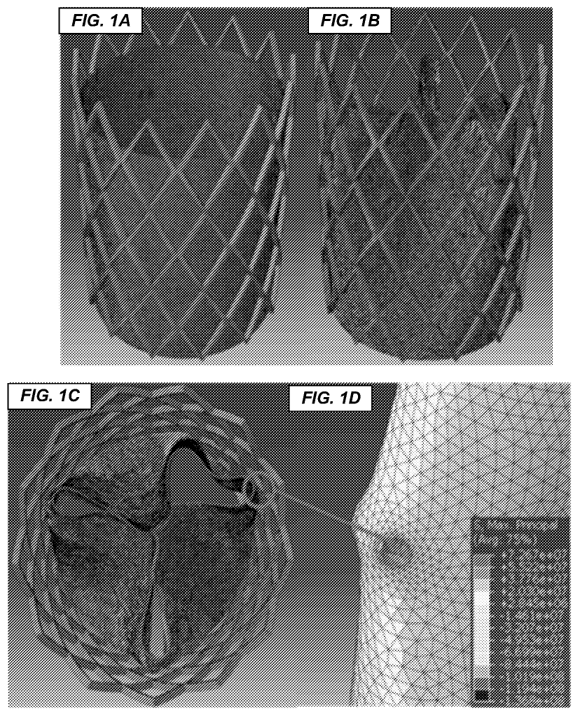

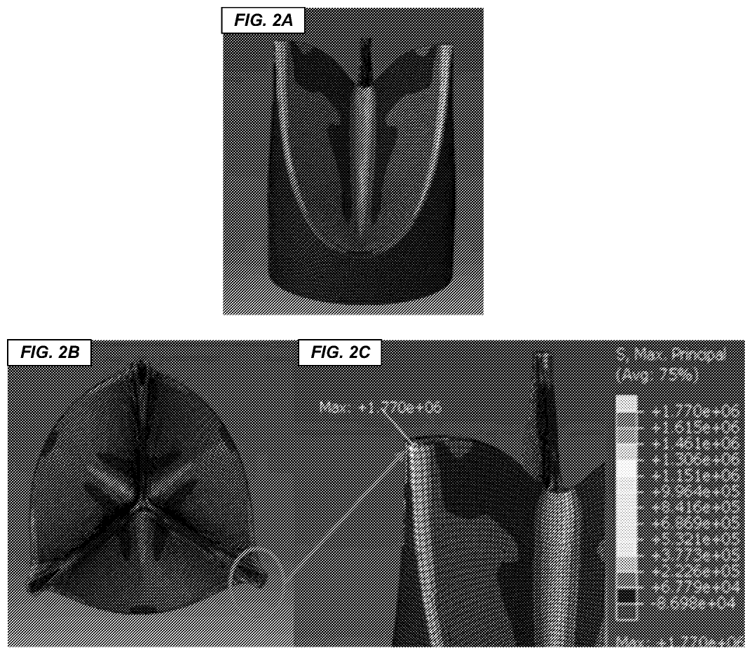

To primarily understand the fundamental loading conditions on the valve and prevent subsequent mechanical wear and tear, computational simulations were conducted to evaluate the efficacy of our previously established design. Two CAD designs were simulated-one from the inventors prior work comprising a simple stent made of Zn-Al4 and a tubular film of polycaprolactone (PCL) (Mn=80,000 Da), and a second design where 3D leaflets anchored more securely to the stent frame were constructed. In the first design (FIG. 1), the PCL film was anchored onto the stent by three sutures on the commissures and circumferentially around the bottom of the film (toward the annular anchor region). The second design comprised 3D leaflets fitted into the same stent and anchored all around the commissures and bottom region shown in FIG. 2. This design modification aimed to assess the loading conditions on each and establish the optimal design. Across all simulations, material properties for PCL were used for the leaflets and applied a load of 25 mmHg (peak fetal pulmonic pressure).

In the first design, peak stresses were located at the sutured points, possibly because, as the valve was crimped in during compression, the anchoring points of the leaflets experienced the greatest loads. Additionally, the leaflets did not coapt completely, which can lead to regurgitation. From the second design, the stresses were observed to be substantially lowered by ˜95% in the commissural region (as shown in FIG. 2), indicating that the 3D leaflet design may yield better results than the tubular film.

From the first design, it was observed that the peak stresses were located at the sutured points, possibly due to the fact that as the valve was pushed in during compression, the leaflets being anchored to those three points experienced the greatest loads. However, as the leaflets were designed as a tubular film, they did not coapt completely. These results can be attributed to the material properties of PCL, hence the leaflet material is simultaneously optimized.

From the second design, it was observed that the stresses were substantially lowered by ˜95% in the commissural region, indicating that the 3D leaflet design may yield better experimental results than the tubular film.

Below is described an exemplary method of fabricating these 3D leaflets on the fetal scale.

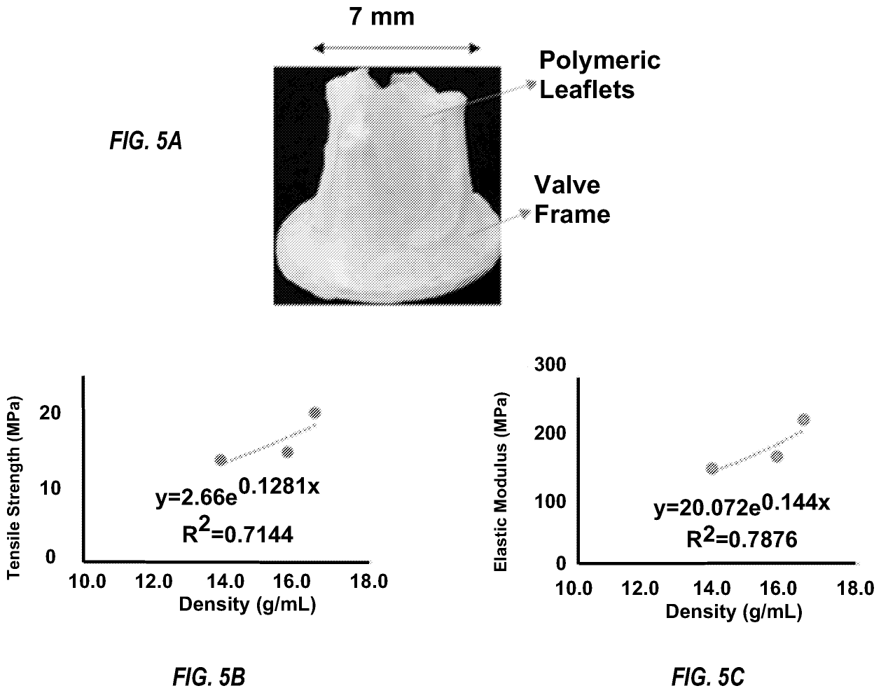

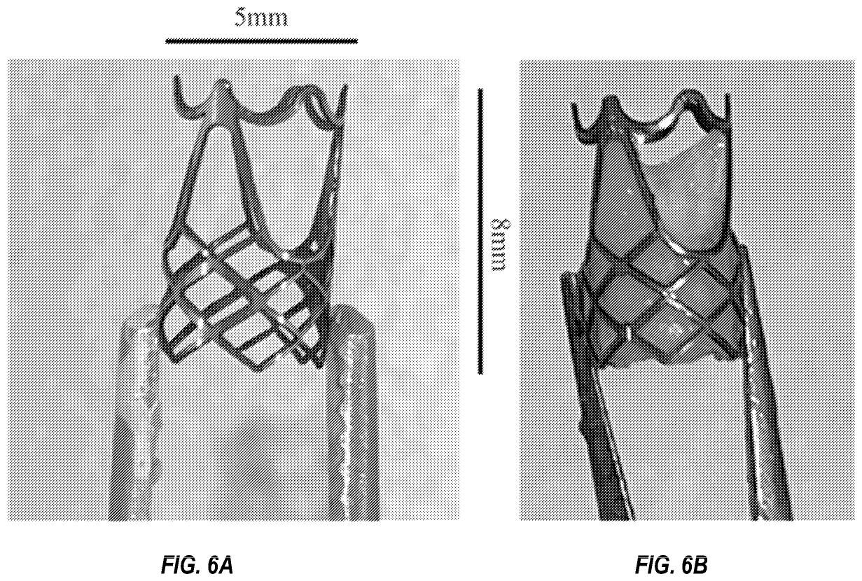

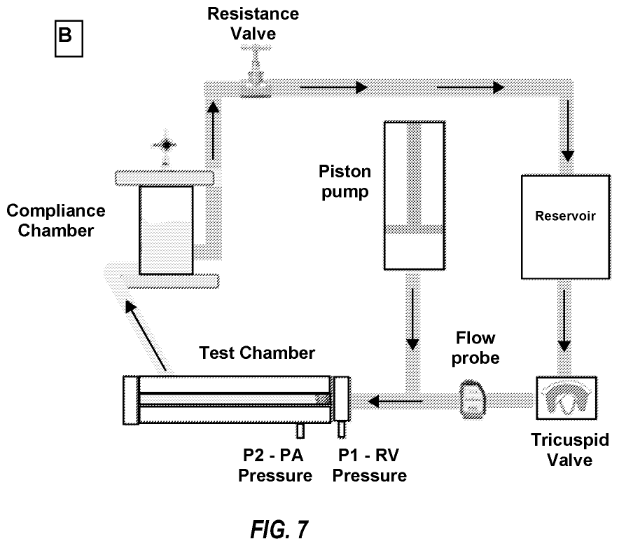

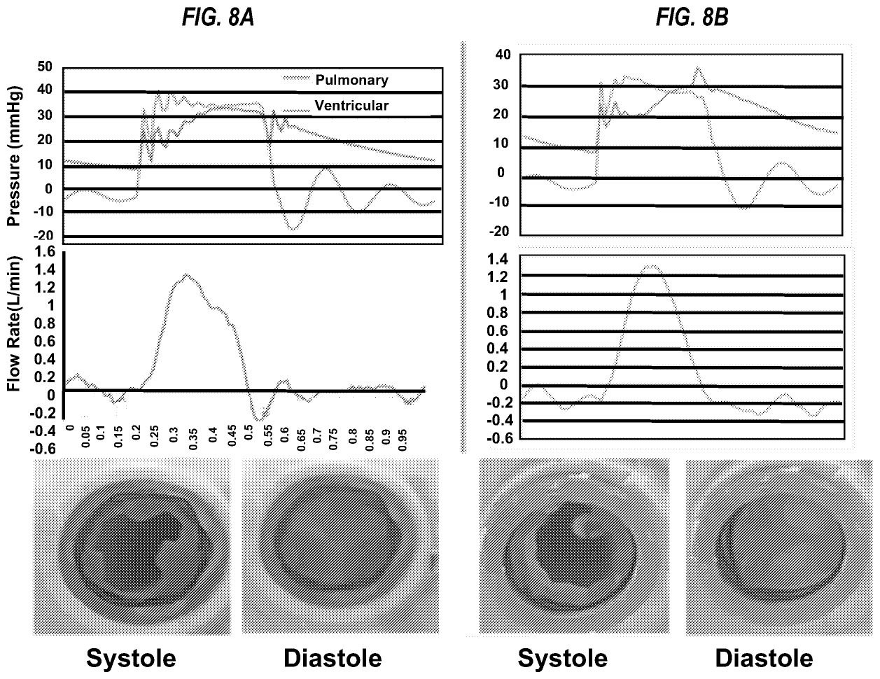

FIGS. 3 & 4 illustrate methods of making a heart valve. In this iteration, the polymeric leaflets were made of polycaprolactone and formed by dipping a mold into a solution containing polycaprolactone and chloroform. The valve frame is made of a 3D printed ABS plastic material. The valve was approximately 7 mm in diameter. The mechanical properties of the polymeric leaflet can be determined based on the regression curves correlating the tensile strength and elastic modulus to the leaflet density. FIG. 6 shows another example in which the polycaprolactone was sprayed rather than dip-coated on the mold. In this example, the valve was approximately 5 mm in diameter. The valve frame was made of a cobalt-chromium stent. The pulsatile flow setup, shown in FIG. 7, was used to assess the hemodynamics of the valve prototypes, as described above. The assembled valves were tested in the right heart simulator with pulmonic pressures 35/8 mmHg in a water-glycerin mixture (40:60 v/v; 99% pure glycerin). The dip-coated valve from FIG. 5A, the effective orifice area (EOA), and the mean gradient were determined to be 0.098 and 10.9 mmHg, respectively. Two different prototypes of FIG. 6 examples were tested, and the results can be seen in FIGS. 8A-B.

The adult size valve design was scaled down (after numerous computational simulations to establish an optimized design) to a fetal size of 5 mm. A 2D drawing was developed from this 3D CAD design in order to laser-cut this design. A tube made of Cobalt-Chromium, specifically the L605 alloy (Vascotube, GmbH, Birkenfield, Germany) was used in this process. The stent was cut using an OPTEC Femtosecond laser (Optec Laser Group, Frameries, Belgium). To manufacture biodegradable resolvable leaflets, 0.75% wt/vol solutions of polycaprolactone (PCL) (Mn=80,000 Da) (Sigma-Aldrich, St. Louis, MO) in chloroform were created. An in-house spray method was used to cast leaflets onto the laser cut stent prototype using 1.2 mL of PCL solution.

As shown in FIG. 7, a custom fetal flow loop was constructed to mimic right heart conditions. The flow loop comprises a reservoir to simulate atrial function, a bladder pump controlled by compressed air that acts as the ventricle, a mechanical valve acting as a tricuspid valve and a pulmonary valve test chamber with a diameter of 7.5 mm, where the prototyped valve was deployed. A compliance chamber was connected in series with a resistance valve to simulate the compliance and resistance of the great arteries. The valve was tested in a working fluid of 60/40 water/glycerin (99% pure glycerin) at 0.5 L/min (cardiac output), 140 beats per minute (heart rate), 70/45 mm Hg (peak systolic/diastolic pulmonary artery pressure) for 100 consecutive cardiac cycles. Transvalvular pressure gradient (PG), regurgitation fraction (RF) and effective orifice area (EOA) were evaluated from this setup.

Homogenous and heterogenous films were cast using the 0.75% wt./v. PCL/chloroform solution. Dried samples underwent accelerated degradation testing using a high-pH solution to understand the natural degradation of the PCL polymer. Samples were weighed to take the pre-degradation mass and then rinsed in dH2O. The samples were then submerged in 8M sodium hydroxide solution at room temperature. Samples were removed from the solution after 20, 30, 40, 60, 120 and 150 minutes. The sample films were rinsed in dH2O and placed in the oven to dry. Pre-degradation masses were compared to the measurements taken after the samples dried to determine the remaining mass.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A method for producing a bioresorbable heart valve comprising:
forming valve leaflets on a first end of at least a portion of a mold coated with a first solution comprising a polymeric material; and
attaching a frame to at least a portion of the coating;
wherein the bioresorbable heart valve is configured to accommodate growth of a patient with the bioresorbable heart valve.

2. The method of claim 1, wherein the bioresorbable heart valve is configured to accommodate growth of the patient being a fetus with the bioresorbable heart valve having been implanted in utero.

3. The method of claim 1 further comprising:
airbrushing the at least a portion of the mold with the first solution;
wherein the valve leaflets comprise one or more commissures having a thickness greater than an average thickness of the valve leaflets.

4. The method of claim 1 further comprising at least one of:
dip coating at least a portion of the mold with the first solution;
spray coating at least a portion of the mold with the first solution; or
airbrushing at least a portion of the mold with the first solution.

5. The method of claim 1, wherein the polymeric material comprises a synthetic polymer selected from a group consisting of polyethers, polyamides, polyurethanes, and polyesters.

6. (canceled)

7. The method of claim 5, wherein the polymeric material further comprises a naturally derived polymer selected from a group consisting of polypeptides, proteins, polysaccharides, glycoproteins, and glycosaminoglycan.

8. (canceled)

9. The method of claim 1, wherein the polymeric material comprises a naturally derived polymer selected from a group consisting of polypeptides, proteins, polysaccharides, glycoproteins, and glycosaminoglycan.

10. (canceled)

11. A method for producing a resorbable heart valve comprising:
providing a first solution comprising a polymeric material comprising at least one biodegradable polymeric material;
coating at least a portion of a mold with the first solution to form a coating;
forming valve leaflets on a first end of the coating;
placing a frame around at least a portion of the coating; and
attaching the frame to the coating;
wherein coating the at least a portion of the mold with the first solution comprises:
spray coating the at least a portion of the mold with the first solution;
airbrushing the at least a portion of the mold with the first solution; or
dip coating the at least a portion of the mold with the first solution.

12. The method of claim 11, wherein:
the coating is a multi-layered coating; and
coating the at least a portion of the mold with the first solution forms a first layer in the multi-layered coating.

13. The method of claim 12 further comprising;
coating the at least a portion of the first layer with a second solution to form a second layer of the multi-layer coating.

14. The method of claim 11, wherein the valve leaflets comprise one or more commissures having a thickness greater than an average thickness of the valve leaflets.

15. The method of claim 11, wherein at least one of:
attaching the frame to the coating comprises suturing the frame to the coating;
attaching the frame to the coating comprises coating at least a portion of the frame with a third solution different than the first solution
the frame is configured as an expandable and contractable stent;
the frame is configured as expandable and contractable stents;
the frame comprises a material selected from a group consisting of plastics, metals, and carbon;
the frame comprises a metal selected from a group consisting of zinc, iron, aluminum, magnesium, nickel, silver, titanium, and alloys thereof; or
the frame comprises a bioresorbable material.

16.-22. (canceled)

23. A resorbable heart valve comprising:
a frame; and
a plurality of leaflets coupled to the frame, the plurality of leaflets comprising a first polymeric material;
wherein the first polymeric material comprises:
a synthetic polymer;
a naturally derived polymer;
a synthetic polymer and a naturally derived polymer; or
at least one biodegradable polymeric material.

24.-28. (canceled)

29. The valve of claim 23, wherein:
the synthetic polymer is selected from a group consisting of polyethers, polyamides, polyurethanes, and polyesters; and
the naturally derived polymer is selected from a group consisting of polypeptides, proteins, polysaccharides, glycoproteins, and glycosaminoglycan.

30. (canceled)

31. The valve of claim 23, wherein:
the plurality of leaflets are multi-layered;
a first layer in the multi-layered leaflets comprises a synthetic polymer; and
a second layer in the multi-layered leaflets comprises a naturally derived polymer.

32.-33. (canceled)

34. The valve of claim 23, wherein the plurality of leaflets comprise one or more commissures having a thickness greater than an average thickness of the plurality of leaflets.

35. The valve of claim 23, wherein the frame is attached to the plurality of leaflets with at least one of:
one or more sutures; or
a second polymeric material different than the first polymeric material.

36.-42. (canceled)

43. A device for use in implanting a valve in a patient comprising:
a cannula having a first end, a second end, and an outer wall defining an internal cavity;
a trocar positioned within the internal cavity of the cannula, the trocar having an outer wall defining an internal cavity; and
a catheter positioned within the internal cavity of the trocar;
wherein at least one of:
the trocar comprises a sharpened tip comprising an opening into the internal cavity of the trocar;
the outer wall of the cannula comprises a polymeric material; or
the outer wall of the trocar comprises a metal.

44. (canceled)

45. The device of claim 43, wherein the opening of the sharpened tip is asymmetrical.

46. The device of claim 43 further comprising;
a pressure transducer configured to monitor a pressure within a portion of the internal cavity of the trocar.

47. The device of claim 46, wherein the pressure transducer is disposed within the internal cavity of the trocar.

48. The device of claim 46 further comprising:
one or more circumferential perforations extending through the outer wall of the trocar.

49.-50. (canceled)
